(19) United States
(12) Patent Application Publication
(10) Pub. No.: US 2025/0073412 A1
(43) Pub. Date: Mar. 6, 2025

(54) DISPOSABLE TRACHEAL CUFF ADAPTER FOR RIGID BRONCHOSCOPE

(71) Applicant: Harold W. Burke, Ridgewood, NJ (US)

(72) Inventor: Harold W. Burke, Ridgewood, NJ (US)

(21) Appl. No.: 18/818,541

(22) Filed: Aug. 28, 2024

Related U.S. Application Data
(60) Provisional application No. 63/535,018, filed on Aug. 28, 2023.

(51) Int. Cl. A61M 16/04; A61B 1/267

(52) U.S. Cl. CPC A61M 16/0434; A61B 1/2676; A61M 16/0463

(57) ABSTRACT

In some embodiments, there may be provided methods, systems, and articles of manufacture for a cuff device configured to be coupled to a rigid bronchoscope. The cuff device may include a first hollow tube comprising a proximal port and a distal port, the proximal port sized with an inner diameter that slides and couples to an exterior surface of a second hollow tube portion of the rigid bronchoscope; a balloon circumscribing at least a portion of the first hollow tube, wherein the balloon is configured to have at least two modes a deflated mode and an inflated mode; and a pilot apparatus comprising a pilot balloon and pilot tubing for inflating the balloon.

### CROSS-REFERENCE TO RELATED APPLICATIONS

This application claims priority to U.S. Provisional Application No. 63/535,018 filed Aug. 28, 2023, entitled “DISPOSABLE TRACHEAL CUFF ADAPTER FOR RIGID BRONCHOSCOPE,” the contents of which is hereby incorporated by reference in its entirety.

### BACKGROUND

Rigid bronchoscopes are often used to visualize airways for diagnostics or therapeutics, for example, to visualize anatomical pathologies, retrieve foreign bodies, or secure the airways. But in some cases, when a physician cannot intubate a patient using conventional methods, the physician may use a rigid bronchoscope to access the patient's trachea.

Rigid bronchoscopes used in this manner may have only a limited ability to ventilate a patient. A rigid bronchoscope has an anesthesia machine outflow connection allowing for ventilation of the patient. But this method only allows for passive oxygenation of the patient, as oxygen and anesthetic gases leak from the bronchoscope, and it produces no pressure to cause expansion of the lungs. Passive oxygenation may be sufficient in patients who can spontaneously ventilate when unconscious. But in some emergent scenarios, passive oxygenation may rapidly become unsatisfactory. For example, passive oxygenation would not be sufficient if a patient were to become apneic while under general anesthesia. Furthermore, should regurgitation of gastric contents occur while in use, the rigid bronchoscope has no inherent means of preventing aspiration.

### SUMMARY

In some example embodiments, there may be provided methods, systems, and articles of manufacture for a cuff device configured to be coupled to a rigid bronchoscope.

In some embodiments, there is provided a cuff device for insertion into a rigid bronchoscope. The cuff device may include a first hollow tube comprising a proximal port and a distal port, the proximal port sized with an inner diameter that slides and couples to an exterior surface of a second hollow tube portion of the rigid bronchoscope; a balloon circumscribing at least a portion of the first hollow tube, wherein the balloon is configured to have at least two modes a deflated mode and an inflated mode; and a pilot apparatus comprising a pilot balloon and pilot tubing for inflating the balloon. The pilot apparatus comprises the pilot balloon coupled to the pilot tubing, which is further coupled to the pilot balloon, wherein the pilot balloon directs gas through the pilot tubing to inflate the balloon. The pilot tubing extends a length of the bronchoscope. The cuff device further comprises one or more safety strings coupled to the first hollow tube to enable removing the cuff device from a subject. The balloon comprises an endotracheal balloon. The balloon is adjacent to the distal port of the first hollow tube. The endotracheal balloon contains up to 10 milliliters (mL) of air. The endotracheal balloon is positioned distal to a trachea of a human subject. The endotracheal balloon when inflated forms at least a partial seal with a trachea to enable active or positive pressure ventilation and to prevent leakage of gases. The endotracheal balloon when inflated forms at least a partial seal to prevent aspiration of regurgitant volume from a stomach into one or more lungs. The first hollow tube comprises a plastic. The plastic is a hard plastic. The hard plastic is composed of one or more of the following: a polyvinyl chloride (PVC), a polyethylene terephthalate (PET), a polyethylene, an acrylonitrile butadiene styrene (ABS), a polycarbonate, a polypropylene, or a polystyrene. The inner diameter of the proximal port of the first hollow tube is sized between 2 and 14 millimeters (mm) to enable sliding and coupling to the exterior surface of the second hollow tube portion of the rigid bronchoscope. The outer diameter of the exterior surface of the second hollow tube portion of the rigid bronchoscope is sized between 2 and 14 mm. The first hollow tube is removable from the second hollow tube portion of the rigid bronchoscope to enable disposable of the cuff device. The hollow tube, balloon, and/or pilot apparatus is disposable.

In some variations, one or more features disclosed herein including one or more of the following features may be implemented as well.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1A illustrates an example of a rigid bronchoscope including a cuff device in operation inside of a human subject, in accordance with some embodiments.

FIG. 1B illustrates another example of a rigid bronchoscope including a cuff device, in accordance with some embodiments.

FIG. 2 illustrates an isometric view of the cuff device, in accordance with some embodiments.

FIG. 3 illustrates an anterior view of the cuff device, in accordance with some embodiments.

FIG. 4 illustrates a lateral view of the cuff device when attached to a rigid bronchoscope, in accordance with some embodiments.

### DETAILED DESCRIPTION

Medical devices used for access to or analysis of an airway may be difficult to manipulate in some types of subjects. For example, children and some adults may have challenging airways (e.g., reduced mouth opening, short thyromental distance, mandibular prognathism, redundant soft tissue, or elongated and elliptical epiglottises).

In many medical situations, a medical team uses a rigid bronchoscope to evaluate a child's airway or another type of small airway. For example, if a child swallows something and chokes (or in other type of emergent situation) a medical team may use a camera attached to the rigid bronchoscope to locate an obstruction to the airway. In another example, a medical team may use a rigid bronchoscope to observe a subject's vocal cords to determine if the subject's snoring is indicative of a medical condition, such as sleep apnea. In these and other cases for example, a medical team may comprise an anesthesiologist to administer anesthesia and incapacitate a subject, while another type of specialist (e.g., an ear, nose, and throat (ENT) surgeon, and/or the like) observes the airway and features of the airway. As a result, the rigid bronchoscope may be connected to an anesthesia circuit that supplies air and anesthetic gases to the subject.

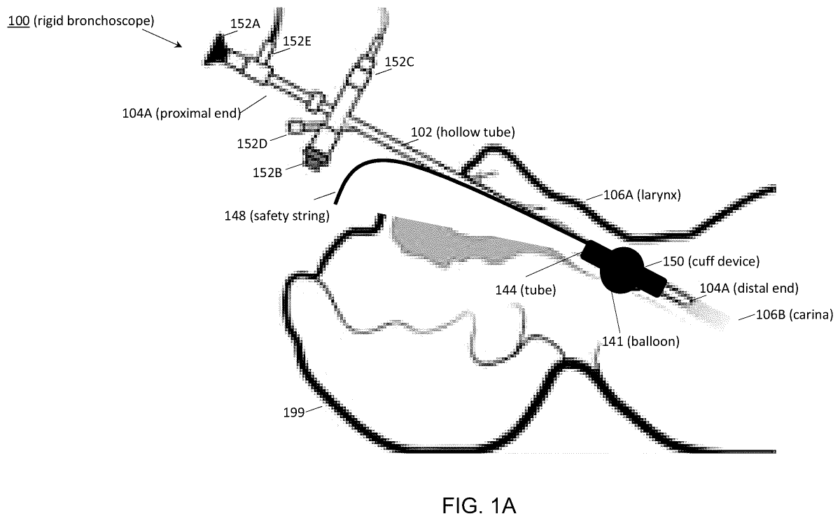

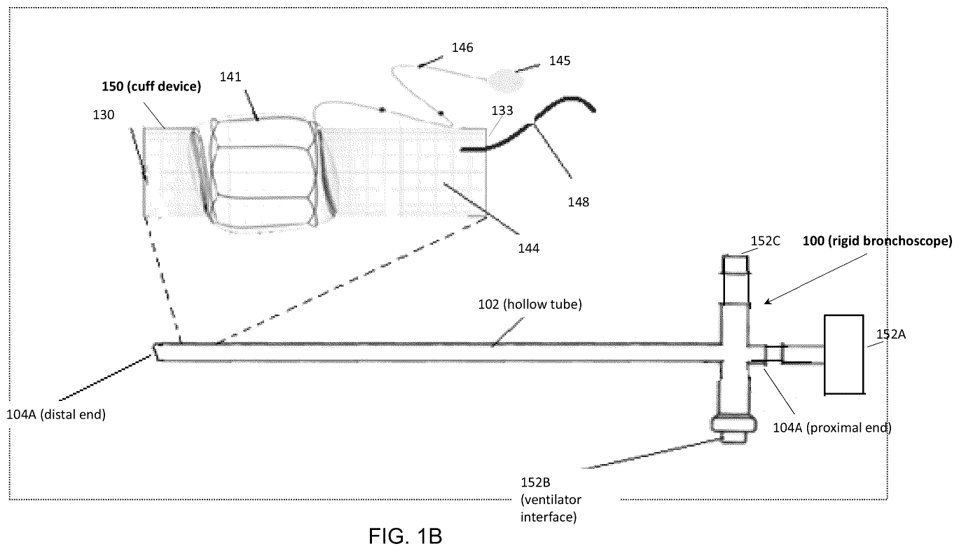

FIG. 1A depicts an example of a rigid bronchoscope 100 in operative use in a subject 199, in accordance with some embodiments. The rigid bronchoscope includes a hollow tube 102 having a distal end 104A and a proximal end 104B. The distal end 104A may be inserted into an opening of the subject, which in the example of FIG. 1A is the mouth and into the trachea between the larynx 106A and the carina 106B. The hollow tube 102 may have, for example, an external diameter between about 2 to 18 millimeters (mm), wall thickness between about 2 to 3 mm, with a length between about 20 mm to 43 mm, although other dimensions may be implemented for the hollow tube. The proximal end 104B may be coupled to one or more attachments, such as visualizing equipment (e.g., a telescope and light source which can be inserted though the hollow tube to visualize the airways), video equipment, accessory instruments (e.g., forceps, suction tubing, and/or other accessories), an anesthesia machine connection (e.g., a connection to an anesthesia circuit), and/or the like.

In the example of FIG. 1A, the rigid bronchoscope 100 includes at the proximal end 104B an endoscope (e.g., camera mounted scope) 152A, a ventilator interface 152B (which can couple to a ventilator circuit), a light source 152C, a suction tubing port 152D, and a anesthesia machine connection 152D. Moreover, the rigid bronchoscope may have grasping forceps configured to grasp soft tissue or foreign bodies. Moreover, the bronchoscope comprises a handle for manipulation of the bronchoscope through the airway. When the subject 199 is under observation (e.g., by a medical team), emergent circumstances may cause the subject to stop breathing, for example. These emergent circumstances may prove fatal if not immediately treated. Although the rigid bronchoscope and anesthesia circuit may provide air and/or gas, (e.g., oxygen and/or anesthetic gases), the subject's lungs may not expand to facilitate the exchange of air. Active, or “positive pressure” ventilation may be needed to completely ventilate, oxygenate, and/or anesthetize the subject. Moreover, while the subject is being ventilated, the subject's stomach may cause gastric content to be aspired into the lungs, but existing rigid bronchoscope assemblies are not configured to mitigate this gastric content aspiration.

In some embodiments, there is provided a cuff device (or “cuff” for short) comprising a balloon that allows active ventilation of a subject (e.g., a patient) when a rigid bronchoscope is used to access the subject's trachea. Moreover, the cuff may mitigate the noted gastric content aspiration. Active ventilation described herein provides a positive pressure that is also a safe amount of ventilation for a patient that is unable to initiate respiration. In accordance with some embodiments, the cuff may be used by, for example, otolaryngologists, anesthesiologists, and other airway experts who require the use of a rigid bronchoscope in patient populations. If a patient is in danger of desaturation during a procedure for example, a physician can quickly inflate the cuff's balloon to secure the patient's airway enable active ventilation to the patient while mitigating gastric content aspiration.

FIG. 1A depicts an example of a cuff device 150 including the balloon 141, which are configured around the hollow tube 102 of the rigid bronchoscope 100, in accordance with some embodiments.

In some embodiments, the cuff device 150 comprises a rigid, hollow tube (e.g., cylinder) 144 circumscribed by the inflatable balloon 141, wherein the cuff device can be inserted into the hollow tube portion 102 of the rigid bronchoscope 100. The cuff device (or cuff for short) converts a rigid bronchoscope into a secure airway device without the use of an endotracheal tube. The cuff may also have one or more safety strings 148 that would also extend the length of the bronchoscope (for example, to enable removal of the cuff in case the cuff were to become dislodged from the bronchoscope).

The cuff 150 is configured to be easily inserted onto the distal end 104A of the rigid bronchoscope 100, used on the subject 199, and when then removed from the rigid bronchoscope 100 when a session with the subject is completed. The inner diameter of the rigid, hollow tube 144 of the cuff may be between 2 and 14 millimeters, for example, to match the outer diameter of the exterior surface of the rigid bronchoscope. As such, the cuff 150 may be considered disposable. Thus for example, during another session requiring use of the rigid bronchoscope, another cuff device can be inserted through the distal end 104A of the rigid bronchoscope 100, used on the subject 199, and then removed from the rigid bronchoscope 100 when the session is completed. In this way, the disposability of the cuff can reduce infections and device integrity failure caused by re-use of the cuff.

Referring to the example of FIG. 1A, the cuff 150 may be configured such that the inner diameter of the cuff is sized to be inserted over the external diameter of hollow tube. To illustrate further, the cuff may be sized with an inner diameter that can accommodate a standard (e.g., pediatric) bronchoscope's external diameter (e.g., external diameters of 4 mm, 5 mm, 6 mm, and/or the like).

The cuff's 150 inflatable balloon 144 may provide active, positive pressure ventilation by for example preventing leakage of oxygen and vital anesthetic gases around the hollow tube 102 of the bronchoscope. In other words, the inflatable balloon portion of the cuff expands and may thus create a barrier or seal around the subjects trachea so oxygen and vital anesthetic gases flow into the subject's lungs. The cuff's balloon 144 may (when inflated) also exert pressure against the trachea and create at least a partial seal between the balloon and the exterior surface of the trachea that causes the oxygen and anesthetic gases to expand the lungs and allows for pulmonary gas exchange. And the cuff's inflatable balloon (when inflated) may, as noted, prevent regurgitant volume from the stomach from aspiration into the lungs, which might otherwise lead to complications such as acute hypoxemia, aspiration pneumonitis, pneumonia, or acute respiratory distress syndrome. The balloon 144 may be heat-sealed to the hollow tube 102.

In operation, the cuff 150 may be used to provide ventilation even in the event of an unknown airway or a known difficult airway, where traditional methods of securing the airway may be unsuccessful. In a known difficult airway, a physician may rapidly deploy the device by attaching the cuff to a rigid bronchoscope to allow ventilation or oxygenation. Even if one dealt with an unknown difficult airway, the cuff may be easier to use than a standard endotracheal tube. This may provide effective when the rigid bronchoscope is manipulated in an airway with narrowed anatomy, such as a bloody, edematous, or pustular airway.

While flexible endotracheal tubes can be used to evaluate some adults, a flexible endotracheal tube may be too long and/or bulky to use in a child or in an adult having a small airway. By contrast, the cuff 150 (which can be inserted and removably affixed to the hollow tube 102 of the rigid bronchoscope) may be easily affixed to a rigid bronchoscope due to the cuff's small form factor. This ease of removal aspect of the cuff (while removable affixed to the rigid bronchoscope) may prove be advantageous when a subject only needs to be ventilated for a short time (e.g., in cases where a physician needs to intervene for a couple of breaths of the subject to prevent hypoxia of the subject) and the placement of a standard endotracheal tube would be unnecessary

As noted, the cuff 150 may be inserted and removably affixed to the rigid bronchoscope (and in particular, the distal end 104A of the hollow tube 102) and may be used, in operation, on a subject, such as the subject 199. The subject may be an adult, a child, an infant, and/or the like. And the subject may be a human subject, although a non-human subject having a trachea (e.g., a dog, pig, ape, chicken, lizard, and/or the like) may be used as well.

FIG. 1B shows a side view of an example implementation of the cuff 150 including balloon 141 and a rigid bronchoscope 100, in accordance with some embodiments. As noted above, the cuff can be slide over the distal end 104A of the hollow tube 102 of the rigid bronchoscope 100. For example, the inner diameter of the cuff may, as noted, be sized so that it can be inserted (and removably affixed) over the external diameter of hollow tube 102. The sizing of the cuff diameter may be configured to prevent the cuff and/or bronchoscope from sliding, providing a tight interchange and secure fit.

In the example of FIG. 1B, the rigid bronchoscope 100 includes the hollow tube 102, the attachment site 152B which can be used to interface to an anesthesia circuit and/or a ventilation circuit, although all of the attachments 152 B, D, and E of FIG. 1A are not depicted. In operation, the anesthesia circuit may provide oxygen, anesthesia, and/or other gases to a subject, such as subject 199, while removing carbon dioxide. The anesthesia circuit may comprise a source of gas, breathing tubing to direct the gas, an adjustable pressure limiting valve to control pressure within the system and direct waste away, a reservoir bag to allow assisted ventilation, and/or the like.

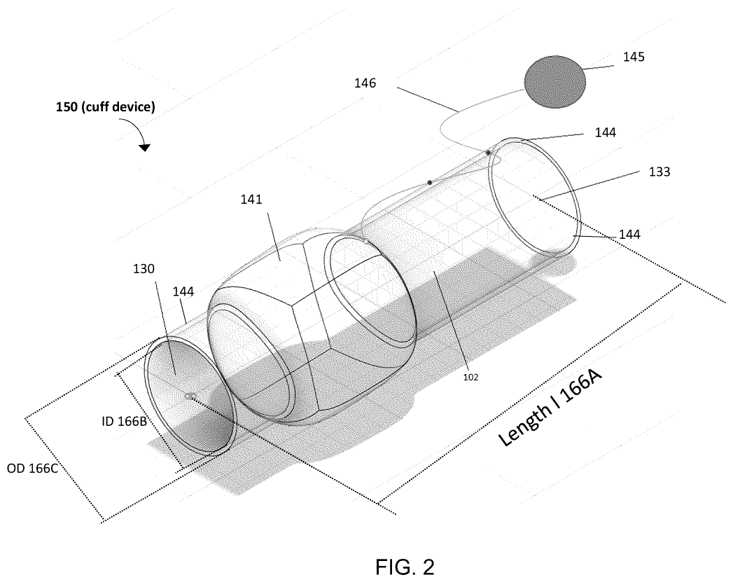

Referring to the cuff 150 including inflatable balloon 141 at FIG. 1B, the cuff comprises a hollow, circular tube (e.g., cylinder) portion 144 having an interior diameter, an exterior diameter, and a length, which are further described with respect to FIG. 2.

FIG. 2 depicts a perspective, top-side view of the cuff 150. Referring to FIGS. 1B and 2, the hollow tube portion 144 includes an internal cavity that is hollow such that the hollow tube 102 can be inserted through the entirety of the cuff's hollow tube portion 144. The cuff's hollow tube portion 144 may have a length 1 166A, an inner diameter 166B, and an outer diameter 166C. For example, the inner diameter may be between 2 and 18 mm, the outer diameter may be between 2.1 mm and 18.1 mm, and the length may be between one centimeter and six centimeters. As noted, the inner diameter 166B is sized such that the distal end 104A of the hollow tube 102 of the rigid bronchoscope can fit into that inner diameter 166B and placed around the hollow tube 102 of the rigid bronchoscope. The cuff may be placed adjacent to the distal end of the rigid bronchoscope, for example, adjacent to the bronchoscope's light source or grasping teeth. The interior of the cuff may have an interior coating, adhesive, or rubber ring configured to secure the bronchoscope inside it.

Manual traction may be used to move the cuff device along the rigid bronchoscope. Hence, the cuff may be positioned at other locations of the bronchoscope than the distal end of the bronchoscope. Placing the cuff device near the camera of the bronchoscope (e.g., at the distal end of the bronchoscope) allows the bronchoscope's camera to be used to determine an exact location in which to insufflate the balloon of the cuff. This may be important when accessing pediatric airways, where large branch points in the lungs may be millimeters apart.

Moreover, the cuff 150 comprises a distal port opening 130 and a proximal port opening (also referred to as port) 133. The proximal port opening 133 serves as an entrance site through which the hollow tube 102 of the rigid bronchoscope 100 can be inserted.

The cuff's hollow tube 144 may be composed of a hard plastic. As used herein, hard plastic refers to a rigid plastic, such as polyvinyl chloride (PVC), polyethylene terephthalate (PET), polyethylene, acrylonitrile butadiene styrene (ABS), polycarbonate, polypropylene, or polystyrene. The hollow tube 144 may be opaque, transparent, or translucent. Moreover, the hollow tube 144 may be inserted just adjacent to a light source attachment (e.g., halogen, incandescent, or light emitting diode (LED)) of the rigid bronchoscope.

The cuff 150 further comprises the balloon 141, such as an endotracheal balloon, which surrounds the exterior surface of a portion of the hollow tube 144. In other words, the balloon 141 circumscribes the hollow tube 144. The balloon 141 may be made of a thin and flexible plastic polymer, such as a silicone elastomer, soft polyvinyl chloride (PVC), PEBAX silicone, thermoplastic elastomer, polyurethane, and/or the like. The balloon 141 may be able to hold several milliliters (e.g., up to 10 mL) of a gas such as sealed air and/or the like.

The balloon 141 may be inflated to a size (e.g., with a max state volume of insufflation from 2 milliliters (mL) to 15 mL) that is configured to enable the cuff device to operate effectively (e.g., block leakage of air or gas, block aspiration of gastric content into the respirator system, and/or allow for active or positive pressure ventilation) in an airway for a subject. For example, for a newborn human subject using a 2 mm bronchoscope, the balloon may be inflated to hold 2 milliliters (mL) of air. But for an adult subject using a 14-18 mm bronchoscope, the balloon may be inflated to hold up to 15 mL.

The balloon 141 may have a size of up to about three times the inner diameter of the hollow tube 144 (e.g., from up to about 6 mm for a 2 mm rigid bronchoscope device or up to about 54 mm for an 18 mm rigid bronchoscope device), when inflated. When not inflated, the balloon 141 may be flush with the outer diameter of the hollow tube (e.g., between 2.1 and 18.1 mm). The balloon 141 may be located closer to the proximal end of the hollow tube 144, closer to the distal end of the hollow tube 144, or in the center of the hollow tube 144. The balloon 141 may be sized to be about ⅓ of the length of the hollow tube 144 (e.g., between about 3 mm and about 2 cm, depending on the length of the hollow tube).

The balloon may be configured to be filled with air sufficient only to enable operation of the device, as overfilling may cause tracheal damage to some subjects, such as infant and child subjects. When the balloon 141 is deflated (e.g., deflated mode), it may be effectively flush with the surface of the cuff device (e.g., configured to not add significantly to the diameter of the hollow tube). The endotracheal balloon 141 may be positioned in a subject's body just distal to and below the voice box or larynx and above the carina, so that the cuff's balloon can provide active ventilation to both of the subject's lungs.

The cuff 150 may also comprise a pilot apparatus for inflating the balloon 141. For example, the pilot apparatus may comprise a pilot balloon 145 coupled to a pilot tubing 146, which further couples to the balloon 141. In this way, the pilot balloon 145 can “pumped” to inflate the balloon 141 when the balloon 141 is inserted into the subject 199 as shown at FIG. 1A. For example, the pilot balloon 105 may be inflated using for example a slip tip syringe (e.g., a 10 cubic centimeter (cc) syringe) to supply air to the balloon via a one-way valve coupled to pilot balloon 105.

The cuff 150 may also comprise one or more safety strings 148 as shown in the example of FIGS. 1A and 1B. The safety string may be used to prevent at least a portion of the bronchoscope or device from passing too far down the trachea of the subject (e.g., enable removal of the cuff if it becomes separated from the rigid bronchoscope). The safety string may be brightly colored to assist with visual acquisition by a physician (although other color schemes or visual indicators may be used additionally or alternatively). For example, the safety string 148 may couple to the cuff and extend out through the subject's mouth to enable retrieval of the cuff 150 (e.g., by pulling the string 148), should the device become dislodged from the rigid bronchoscope. The safety string may for example be secured (via hemostats or other external fasteners) to a bed, a towel, and/or the like.

In some embodiments, the cuff 150 may, as noted, have components which are disposable and intended for a single use, while the rigid bronchoscope 100 is not disposable and intended for multiple uses. For example, the cuff's inflatable balloon, pilot apparatus comprising the pilot balloon and pilot tubing, the hollow tube, safety strings, or any combination thereof).

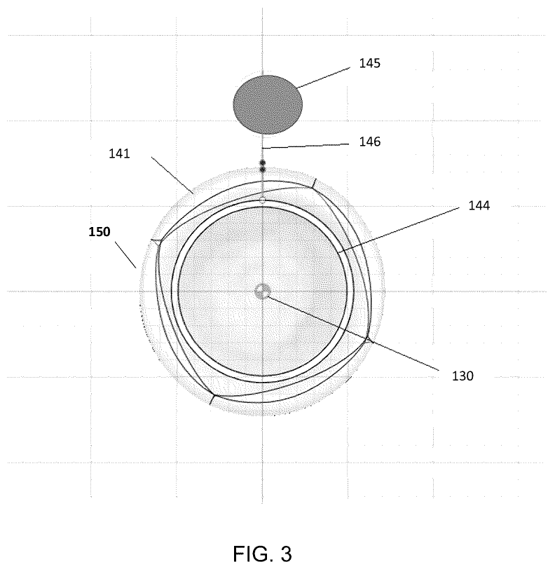

FIG. 3 illustrates a front view of the cuff 150, in accordance with some embodiments. The front view illustrates the cuff's distal port 130, the hollow tube portion 144, and the cuff's balloon 141. Also shown are the pilot balloon 145 and pilot tubing 146.

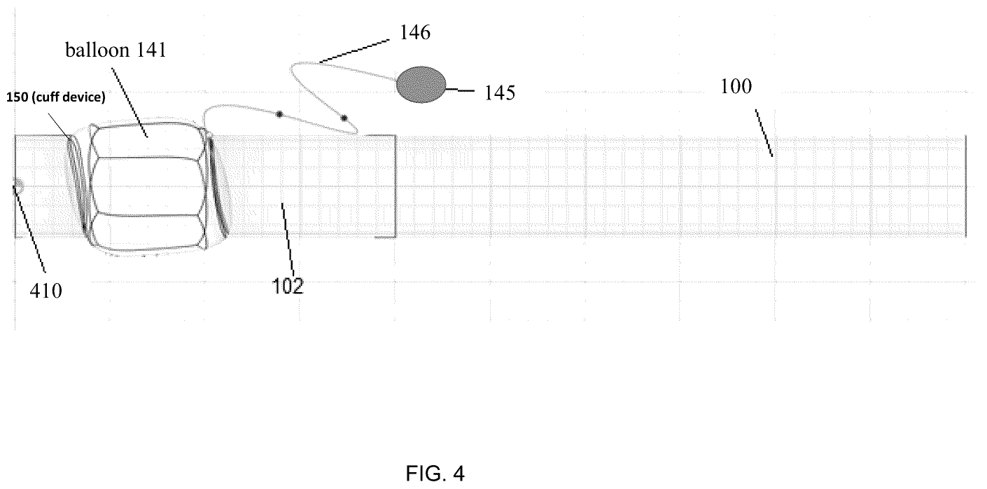

FIG. 4 illustrates a side view of the cuff 150 when attached to the hollow tube 102 of the rigid bronchoscope 100, in accordance with some embodiments. FIG. 4 also shows an accessory, such as a light source 410, at the distal end of the rigid bronchoscope 100.

In an example use case of the cuff 150, the proximal end 133 of the cuff is inserted into the rigid bronchoscope 100 just adjacent to the bronchoscope's light source 410, for example. After a teeth guard is placed on a subject's upper or lower jaw, the rigid bronchoscope may be inserted through the subject's oropharynx while the subject is in, for example, a supine position. The rigid bronchoscope may be manipulated through the hypopharynx, through the vocal cords at the glottis aperture, and into the trachea. The device's balloon 141 may be in a deflated state during this insertion and manipulation process to facilitate the smooth passage of the device through the subject's airway.

If, while the balloon 141 is distal to the subject's vocal cords, the subject were to decompensate unexpectedly (e.g., via hypoxemia, hypercarbia, or unexpected aspiration), the inflatable balloon 141 may be inflated via for example the pilot balloon 145. The pilot balloon may be external to the airway and hold a large enough volume of air to safely occlude the trachea, allowing for positive pressure ventilation with the anesthesia machine connected to the rigid bronchoscope. The pilot balloon 145 and pilot tubing 146 may be configured to extend the length of the rigid bronchoscope, to enable their use regardless of how deeply the bronchoscope is positioned in the subject's airway.

The rigid bronchoscope 150 may be deployed in a known difficult airway, which can occur often in, for example, a pediatric population. A known difficult airway may comprise any clinical situation in which a conventionally trained anesthesiologist may experience difficulty with face mask ventilation of an upper airway, difficulty with tracheal intubation, or both. If the bronchoscope were to be inserted into an edematous airway of a subject who subsequently were to enter apnea or become under neuromuscular blockade, passive ventilation of the subject may be insufficient. But if the cuff were placed on the bronchoscope before insertion, the subject would be able to be actively ventilated. Unlike a standard endotracheal tube, the cuff 150 may be used to enable application of a positive pressure ventilation as noted (e.g., airways that may already be edematous, pustular, or hemorrhagic). If the operation does not require prolonged mechanical ventilation (e.g. foreign body removal) then the rigid bronchoscope 150 may be withdrawn without oral tracheal tube extubation. Yet the use of the cuff 150 to secure a difficult airway does not preclude the use of a cook exchange catheter for conversion to a standard oral endotracheal airway should circumstances require it. In some embodiments, the endotracheal balloon contains up to 10 milliliters (mL) of air.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. For example, the logic flows may include different and/or additional operations than shown without departing from the scope of the present disclosure. One or more operations of the logic flows may be repeated and/or omitted without departing from the scope of the present disclosure. Other implementations may be within the scope of the following claims.

What is claimed:

1. A cuff device for insertion into a rigid bronchoscope, comprising:
a first hollow tube comprising a proximal port and a distal port, the proximal port sized with an inner diameter that slides and couples to an exterior surface of a second hollow tube portion of the rigid bronchoscope;
a balloon circumscribing at least a portion of the first hollow tube, wherein the balloon is configured to have at least two modes: a deflated mode, and an inflated mode; and
a pilot apparatus comprising a pilot balloon and pilot tubing for inflating the balloon.

2. The cuff device of claim 1, wherein the pilot apparatus comprises the pilot balloon coupled to the pilot tubing, which is further coupled to the pilot balloon, wherein the pilot balloon directs gas through the pilot tubing to inflate the balloon.

3. The cuff device of claim 2, wherein the pilot tubing extends a length of the rigid bronchoscope.

4. The cuff device of claim 1, further comprising: one or more safety strings coupled to the first hollow tube to enable removing the cuff device from a subject.

5. The cuff device of claim 4, wherein the one or more safety strings extend to a length of the rigid bronchoscope.

6. The cuff device of claim 1, wherein the balloon comprises an endotracheal balloon.

7. The cuff device of claim 6, wherein the balloon is adjacent to the distal port of the first hollow tube.

8. The cuff device of claim 6, wherein the endotracheal balloon contains up to 10 milliliters (mL) of air.

9. The cuff device of claim 6, wherein the endotracheal balloon is positioned distal to a trachea of a human subject.

10. The cuff device of claim 6, wherein the endotracheal balloon when inflated forms at least a partial seal with a trachea to enable active or positive pressure ventilation and to prevent leakage of gases.

11. The cuff device of claim 6, wherein the endotracheal balloon when inflated forms at least a partial seal to prevent aspiration of regurgitant volume from a stomach into one or more lungs.

12. The cuff device of claim 1, wherein the first hollow tube comprises a plastic.

13. The cuff device of claim 12, wherein the plastic is a hard plastic, wherein the hard plastic is composed of a polyvinyl chloride (PVC), a polyethylene terephthalate (PET), a polyethylene, an acrylonitrile butadiene styrene (ABS), a polycarbonate, a polypropylene, and/or a polystyrene.

14. The cuff device of claim 1, wherein the inner diameter of the proximal port of the first hollow tube is sized between 2 and 18 millimeters (mm) to enable sliding and coupling to the exterior surface of the second hollow tube portion of the rigid bronchoscope, wherein an outer diameter of the exterior surface of the second hollow tube portion of the rigid bronchoscope is sized between 2 and 18 mm.

15. The cuff device of claim 1, wherein the first hollow tube is removable from the second hollow tube portion of the rigid bronchoscope to enable disposable of the cuff device.

16. The cuff device of claim 1, wherein the hollow tube, balloon, and/or pilot apparatus is disposable.
